(19) United States
(12) Patent Application Publication
(10) Pub. No.: US 2023/0295574 A1
(43) Pub. Date: Sep. 21, 2023

(54) GINGIVAL TISSUES AND METHODS OF PREPARATION THEREOF

(71) Applicant: National University of Singapore, Singapore (SG)

(72) Inventors: Sriram Gopu, Singapore (SG); Giridharan Muniraj, Singapore (SG)

(21) Appl. No.: 18/016,570

(22) PCT Filed: Jul. 16, 2021
(86) PCT No.: PCT/SG2021/050418

§ 371 Date: Jan. 17, 2023

(30) Foreign Application Priority Data
Jul. 17, 2020 (SG) 10202006843S

(51) Int. Cl. C12N 5/077; C12N 5/071; G01N 33/50

(52) U.S. Cl. CPC C12N 5/0656; C12N 5/0629; G01N 33/5088; C12N 2537/10; C12N 2501/998; C12N 2513/00

(57) ABSTRACT

The present invention relates to a method of preparing a three-dimensional (3D) cell composition comprising the steps of a) forming a support matrix containing oral fibroblasts suspended within the support matrix by mixing fibrinogen, a modifier and oral fibroblasts with thrombin, b) incubating the support matrix in a cell culture media for a sufficient time to allow development of a first layer of the three-dimensional cell composition, and c) seeding oral keratinocytes on a surface of the first layer and culturing the oral keratinocytes to form a second layer of the three-dimensional cell composition. In specific embodiments of the invention, the method is exemplified for the production of an artificial gingival tissue, wherein polyethylene oxide), 4-arm, succinimidyl glutarate terminated is used as the modifier. Also disclosed are uses of the 3D cell composition which include treatment of a gum disease or condition, regenerative therapy and for in vitro testing.

### FIELD OF INVENTION

The invention relates generally to the field of tissue engineering. In particular, the disclosure teaches a method of preparing a three-dimensional (3D) cell composition and uses thereof of the 3D cell composition.

### BACKGROUND

Gingival tissues or commonly referred to as ‘gums’, are the tissues that cover the tooth and has various functions such as barrier to mechanical, chemical and microbial agents and hence, protect the underlying tissues. The gingival tissues are composed of mucosal connective tissue called lamina propria and an overlying epithelium. The lamina propria is composed of cells (primarily fibroblasts) and blood vessels embedded within a collagenous matrix. The gingival epithelium is composed primarily of keratinocytes arranged as a stratified layer of cells with an outermost cornified or keratinized layer. The stratified layers of the gingival epithelium include basal, spinous, granular and corneal layers. The gingival epithelium and the lamina propria are glued together by a layer of basement membrane. The basement membrane is a thin membrane consisting primarily of collagen type IV, laminins, integrins and fibronectin.

Advances in tissue engineering such as three-dimensional organotypic culture provides opportunities to reconstruct the human tissues in the lab. Using the three-dimensional culture techniques, it possible to reconstruct gingival tissues in vitro. Gingival tissue equivalents are lab-made tissues that can help replace the damaged human gingiva, and has also has crucial applications as an experimental model for testing the safety, toxicity and efficacy of dental and oral-care products, drug screening and investigation of host-microbiome interactions. Hence, the development and fabrication of human gingival tissue equivalents that mimic the structure and physiology of native human gingiva is required for such applications. Since the EU ban on use of animal models for testing of cosmetic products (that includes the oral-care products), various companies have developed reconstructed human gingival epithelium (RHGE) models such as the EpiGingival™ (MatTek) and SkinEthic HGE™ (Episkin). However, these models are based on reconstruction of the gingival epithelium only and lack the underlying connective tissue (lamina propria).

Accordingly, it is generally desirable to overcome or ameliorate one or more of the above mentioned difficulties.

### SUMMARY

Disclosed herein is a method of preparing a three-dimensional cell composition, the method comprising the steps of a) forming a support matrix containing oral fibroblasts suspended within the support matrix by mixing fibrinogen, a modifier and oral fibroblasts with thrombin; b) incubating the support matrix in a cell culture media for a sufficient time to allow development of a first layer of the three-dimensional cell composition; and c) seeding oral keratinocytes on a surface of the first layer and culturing the oral keratinocytes to form a second layer of the three-dimensional cell composition.

Disclosed herein is a three-dimensional cell composition obtained according to a method as defined herein.

Disclosed herein is a three-dimensional cell composition comprising a) a first layer comprising a PEG-fibrin support matrix containing oral fibroblasts suspended within the support matrix, wherein the support matrix comprises fibrin, a modifier and oral fibroblasts; and b) a second layer comprising oral keratinocytes.

Disclosed herein is a three-dimensional cell composition as defined herein for use as a medicament.

Disclosed herein is the use of a three-dimensional cell composition as defined herein in the manufacture of a medicament for treating a gum disease or condition.

Disclosed herein is the use of a three-dimensional cell composition as defined herein in the manufacture of a medicament for regenerative therapy.

Disclosed herein is the use of a three-dimensional cell composition as defined herein for in vitro testing.

### BRIEF DESCRIPTION OF DRAWINGS

Embodiments of the present invention are hereafter described, by way of non-limiting example only, with reference to the accompanying drawings in which:

FIG. 1: Gingival tissue equivalents within 6-well (left) and 12-well (right) insert formats. The dotted circles demonstrate that the gingival tissue equivalents fabricated using fibrin-based matrix (and cultured for −3 weeks) occupy the entire area of the insert without any contraction.

FIG. 2. Hematoxylin-Eosin stained photomicrographs of the gingival equivalents (Gingiva-FT) demonstrating the presence of keratinized stratified squamous epithelium consisting of the basal, spinous, granular and cornified layers on a gingival fibroblast-populated lamina propria matrix.

FIG. 3. Photomicrographs of the gingival equivalents (Gingiva-FT) demonstrate the expression of cytokeratins (CK-5 and CK-10).

FIG. 4. Photomicrographs of the gingival equivalents (Gingiva-FT) demonstrate the expression of gingival epithelial differentiation markers (Loricrin, Filaggrin and CK-10).

FIG. 5. Photomicrographs of the gingival equivalents (Gingiva-FT) demonstrate the expression of extracellular matrix proteins of the lamina propria (Collagen-1 and Fibronectin).

FIG. 6. Photomicrographs of the gingival equivalents (Gingiva-FT) demonstrate the expression of basement membrane proteins (Collagen-IV and Laminin-5).

FIG. 7. Formation of microvascular networks in the vascularized lamina propria equivalents fabricated using varying concentrations of fibrinogen (Fbrgn: 5 mg/ml, 7.5 mg/ml, 10 mg/ml) and thrombin (Thr: 12UN/ml, 6UN/ml, 3UN/ml, 1.5UN/ml)

FIG. 8. Kinetics of formation of microvascular networks within the vascularized lamina priopria equivalents fabricated using varying concentration of endothelial cells (1.5×10⁶ and 0.75×10⁶ cells) and gingival fibroblasts (5×10⁴ and 10×10⁴ cells) over 8 days of culture.

FIG. 9. Hematoxylin-Eosin stained photomicrographs of the vascularized gingival equivalents (Gingiva-FT-V).

FIG. 10. Photomicrographs of the vascularized gingival equivalents (Gingiva-FT-V) demonstrate the expression of cytokeratins (CK-5 and CK-10).

FIG. 11. Photomicrographs of the vascularized gingival equivalents (Gingiva-FT-V) demonstrate the expression of gingival epithelial differentiation markers (Loricrin, Filaggrin and CK-10).

FIG. 12. Photomicrographs of the vascularized gingival equivalents (Gingiva-FT-V) demonstrate the expression of extracellular matrix proteins of the lamina propria (Collagen-1 and Fibronectin).

FIG. 13. Photomicrographs of the vascularized gingival equivalents (Gingiva-FT-V) demonstrate the expression of basement membrane proteins (Collagen-IV and Laminin-5) and endothelial markers (CD31 and vWF). All the four markers also demonstrate the presence of blood vessels in the lamina propria.

FIG. 14. Use of intact Gingiva-FT tissue constructs for investigating the mucosal irritation and barrier disruption potential of mouthwash.

FIG. 15. Use of lamina propria equivalents representative of ulcerated oral mucosa for investigating the mucosal irritation potential of mouthwash on oral ulcers.

FIG. 16. Use of Gingiva-FT tissue constructs for investigating the mucosal corrosion potential of mouthwash exposure over 3 and 60 minutes.

FIG. 17. Use of Gingiva-FT tissue constructs for investigating the dental anaesthetic permeation through intact and SLS-treated tissue constructs.

FIG. 18. Use of Gingiva-FT tissue constructs for investigating the biocompatibility (acute toxicity) of dental composites.

FIG. 19. Use of biofabrication methods to fabricate young and aged oral mucosal phenotypes and their application for oral mucosal ageing studies.

FIG. 20. Use of Gingiva-FT tissue constructs to fabricate young and aged phenotypes of gingival tissues and their application for gingival ageing studies.

### DETAILED DESCRIPTION

The present disclosure teaches a method of preparing a three-dimensional cell composition. The method may comprise a) forming a support matrix containing oral fibroblasts suspended within the support matrix. The method may then comprise b) incubating the support matrix in a cell culture media for a sufficient time to allow development of a first layer of the three-dimensional cell composition. The method may further comprise c) seeding oral keratinocytes on a surface of the first layer and culturing the oral keratinocytes to form a second layer of the three-dimensional cell composition. The support matrix may, for example, be a fibrin-based matrix.

Disclosed herein is a method of preparing a three-dimensional cell composition, the method comprising the steps of a) forming a support matrix containing oral fibroblasts suspended within the support matrix by mixing fibrinogen, a modifier and oral fibroblasts with thrombin; b) incubating the support matrix in a cell culture media for a sufficient time to allow development of a first layer of the three-dimensional cell composition; and c) seeding oral keratinocytes on a surface of the first layer and culturing the oral keratinocytes to form a second layer of the three-dimensional cell composition.

The three-dimensional cell composition may be a three-dimensional cell culture. In one embodiment, the three-dimensional cell composition is a three-dimensional tissue equivalent. The three-dimensional cell composition may comprise two layers, i.e. a first and a second layer.

The three-dimensional tissue equivalent may also be referred to as a full-thickness gingival equivalent comprising a lamina propria equivalent layer as the first layer and a gingival epithelial equivalent layer as the second layer. In one embodiment, the first layer is a lamina propria equivalent layer. In one embodiment, the second layer is a gingival epithelial equivalent layer. Without being bound by theory, it is the complex interaction between the oral fibroblasts and oral keratinocytes that ultimately enables to generation of full-thickness gingival tissue equivalent.

In one embodiment, the three-dimensional cell composition is an artificial gingival tissue.

In one embodiment, the three-dimensional cell composition is an artificial vascularized gingival tissue.

The three-dimensional equivalent may also be referred to as a full-thickness oral mucosal equivalent. In one embodiment, the three-dimensional cell composition is an artificial oral mucosal tissue. Without being bound by theory, it is the complex interaction between the oral fibroblasts and oral keratinocytes that ultimately enables to generation of full-thickness oral mucosal equivalent

In one embodiment, the oral fibroblasts are from gingival, periodontal ligament, buccal mucosa, palatal mucosa, labial mucosa, lingual mucosa or other oral mucosal surfaces.

The term “support matrix” as used herein, refers to any three-dimensional structure made of any material and having any shape and internal structure that allows cells to grow within the three-dimensional structure in more than one layer.

The support matrix may be formed from any suitable material or combination of materials. Examples of suitable materials for forming the support matrix include fibrin, collagen, gelatin, hyaluronan, chondroitin sulfate, alginate, nitrocellulose, carboxymethylcellulose, polyglycolic acid (PGA), polyethylene glycol (PEG) poly(lactic-co-glycolic acid) (PLGA), poly-L-lysine, Matrigel® compositions, poly(lactic acid) (PLA), any suitable synthetic biomaterial, and variations and combinations thereof. In an exemplary embodiment, the support matrix mimics the structure of in vivo extracellular matrices.

In one embodiment, the cells are homogenously suspended in the support matrix.

In one embodiment, the support matrix is formed by mixing fibrinogen, a modifier, oral fibroblasts with thrombin.

In one embodiment, the fibrinogen is cross-linked in the presence of thrombin. In one embodiment, the mixture of fibrinogen and modifier is cross-linked in the presence of thrombin. The cross-linking may form a modifier/fibrin-based support matrix. The fibrin-based support matrix may have a porous three-dimensional structure for oral fibroblasts and other cells to grow with the structure.

In one embodiment, the concentration of fibroblasts is from 1×10⁴ to 1×10⁶ cells/ml (e.g. in terms of number of cells per ml of support matrix). In one embodiment, the concentration of fibroblasts is from 3×10⁴ to 7×10⁵ cells/ml. This concentration of fibroblasts is kept at this level as a higher number of fibroblasts may cause the matrix to be degraded rapidly. In some embodiments, the concentration of the fibroblasts is from 1×10⁴, 2×10⁴, 3×10⁴, 4×10⁴, 5×10⁴, 6×10⁴, 7×10⁴, 8×10⁴ 9×10⁴, 1×10⁵, 1.5×10⁵, 2×10⁵, 2.5×10⁵, 3×10⁵, 3.5×10⁵, 4×10⁵, 4.5×10⁵, 5×10⁵, 5.5×10⁵, 6×10⁵, 6.5×10⁵, 7×10⁵, 8×10⁵, 9×10⁵ or 1×10⁶ cells/ml. The fibroblasts may be of human origin.

The support matrix may be vascularized. In one embodiment, the support matrix further comprises endothelial cells. The endothelial cells may be at a concentration of 1×10⁵ to 4×10⁶ cells/ml (e.g. in terms of number of cells per ml of support matrix). The endothelial cells may be at a concentration of 1.8×10⁵ to 3.75×10⁶ cells/ml. In some embodiments, the concentration of the endothelial cells is from 1×10⁵, 1.5×10⁵, 1.8×10⁵, 2×10⁵, 2.5×10⁵, 3×10⁵, 3.5×10⁵, 4×10⁵, 4.5×10⁵, 5×10⁵, 5.5×10⁵, 6×10⁵, 6.5×10⁵, 7×10⁵, 7.5×10⁵, 8×10⁵, 8.5×10⁵, 9×10⁵, 9.5×10⁵, 1×10⁶, 1.5×10⁶, 2×10⁶, 2.5×10⁶, 3×10⁶, 3.5×10⁶, 3.75×10⁶ or 4×10⁶ cells/ml. The endothelial cells may be of arterial, venous or microvascular origin. The endothelial cells may be of human origin.

In one embodiment, there is provided a method of preparing a three-dimensional cell composition, the method comprising the steps of a) forming a support matrix containing oral fibroblasts and endothelial cells suspended within the support matrix by mixing fibrinogen, a modifier, oral fibroblasts and endothelial cells with thrombin; b) incubating the support matrix in a cell culture media for a sufficient time to allow development of a first layer of the three-dimensional cell composition; and c) seeding oral keratinocytes on a surface of the first layer and culturing the oral keratinocytes to form a second layer of the three-dimensional cell composition.

In one embodiment, the fibrinogen is human fibrinogen.

In one embodiment, the concentration of fibrinogen is 1.25 to 20 mg/ml. The concentration of fibrinogen may, for example, be 1.25, 1.5, 1.75, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 mg/ml.

In one embodiment, the modifier is 0.3 to 2.5 mg/ml. The concentration of the modifier may, for example, be 0.3, 0.4, 0.5, 0.6, 0.7, 0.8, 0.9, 1.0, 1.1, 1.2, 1.3, 1.4, 1.5, 1.6, 1.7, 1.8, 1.9, 2, 2.1, 2.2, 2.3, 2.4 or 2.5 mg/ml. Advantageously, the modifier may increase the cross-linking density of the resultant gel which may improve the mechanical strength, porosity and slow down the degradation of fibrin in the matrix.

In one embodiment, the concentration of fibrinogen is 1.25 to 10 mg/ml and the concentration of the modifier is 0.3 to 2.5 mg/ml.

The weight ratio of fibrinogen to modifier may be about 2:1, about 3:1, about 4:1, about 5:1 or about 6:1. In one embodiment, the weight ratio of fibrinogen to modifier is about 4:1. This ratio of fibrinogen to modifier may improve the mechanical properties, porosity and slow down the degradation of the matrix without significantly affecting cell growth.

The modifier may cross-link different fibrinogen molecules. In one embodiment, the modifier is a 2-arm, 4-arm or 8-arm polyethylene glycol (PEG). In one embodiment, the modifier is a 4-arm PEG. Each 4-arm PEG may cross-link up to 4 fibrinogen molecules. The 4-arm PEG may be poly(ethylene oxide), 4-arm, succinimidyl glutarate terminated.

The thrombin may be provided at a concentration of 1 IU/ml to 15 IU/ml. The thrombin may be provided at a concentration of 3.125 IU/ml to 12.5 IU/ml. The thrombin may, for example, be provided at a concentration of 1, 1.25, 1.5, 1.75, 2, 2.25, 2.5, 2.75, 3, 3.125, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75 or 14 IU/ml.

In one embodiment, the thrombin is human thrombin.

In one embodiment, the step of forming the support matrix comprises forming the support matrix in a mold.

In one embodiment, step c) comprises incubating the support matrix in a media supplemented with fetal bovine serum (e.g. 0.5%), L-ascorbic acid (e.g. 10-100 ug/ml), hydrocortisone (e.g. 50-500 ng/ml), basic fibroblast growth factor (e.g. 1-20 ng/ml), selenium (e.g. 1-10 ng/ml), ethanolamine (e.g. 1-10 ug/ml), insulin (e.g. 1-20 ug/ml), transferrin (e.g. 1-10 ug/ml) and aprotinin (e.g. 10-100 KIU/ml) (see, for example, Media-GE1). The media may further comprise VEGF (e.g. 5-50 ng/ml) and EGF (e.g. 1-10 ng/ml).

In one embodiment, step c) comprises incubating the support matrix in a media supplemented with human serum (e.g. 0.5-5%), human plasma lysate (0.5-5%), human serum albumin (0.5-5%), L-ascorbic acid (e.g. 10-100 ug/ml), hydrocortisone (e.g. 50-500 ng/ml), basic fibroblast growth factor (e.g. 1-20 ng/ml), selenium (e.g. 1-10 ng/ml), ethanolamine (e.g. 1-10 ug/ml), insulin (e.g. 1-20 ug/ml), transferrin (e.g. 1-10 ug/ml) and aprotinin (e.g. 10-100 KIU/ml) (see, for example, Media-GE1). The media may further comprise VEGF (e.g. 5-50 ng/ml) and EGF (e.g. 1-10 ng/ml) (see, for example, Media-VGE1).

In one embodiment, the method comprises incubating the support matrix for 2 to 6 days to form the first layer of the three-dimensional cell composition. The oral fibroblasts in the support matrix may secrete various ECM proteins that eventually gives rise to lamina propria equivalent.

In one embodiment, the method further comprises seeding oral keratinocytes onto a surface of the first layer (i.e. on top of the first layer) and culturing the oral keratinocytes to form a second layer.

In one embodiment, the seeding density of oral keratinocytes is from 1×10⁵ to 5×10⁵ cells/cm² (e.g. number of cells per square cm of the first layer of 3D cell composition). The seeding density oral keratinocytes may be about 1×10⁵, 1.5×10⁵, 2×10⁵, 2.5×10⁵, 3×10⁵, 3.5×10⁵, 4×10⁵, 4.5×10⁵ or 5×10⁵ cells/cm². In one embodiment, the seeding density of oral keratinocytes is from 2.5×10⁵ to 3.5×10⁵ cells/cm².

In one embodiment, the oral keratinocytes may be from gingival, periodontal ligament, buccal mucosa, palatal mucosa, labial mucosa, lingual mucosa or other oral mucosal surfaces. The keratinocytes may be of human origin.

The oral keratinocytes may be cultured in a media comprising a 1:1 mix of endothelial serum-free media (ESFM) and keratinocyte serum-free media (KSFM). The media may be supplemented with 0.5% fetal bovine serum (1-BS), L-ascorbic acid (e.g. 10-100 ug/ml), hydrocortisone (e.g. 50-500 ng/ml), EGF (e.g. 1-10 ng/ml), selenium (e.g. 1-10 ng/ml), ethanolamine (e.g. 1-10 ug/ml), insulin (e.g. 1-20 ug/ml), transferrin (e.g. 1-10 ug/ml) and aprotinin (e.g. 10-100 KIU/ml). The media may further comprise VEGF (e.g. 5-50 ng/ml).

The oral keratinocytes may be cultured in a media comprising a 1:1 mix of endothelial serum-free media (ESFM) and keratinocyte serum-free media (KSFM). The media may be supplemented with human serum (0.5-5%), human plasma lysate (0.5-5%), human serum albumin (0.5-5%), L-ascorbic acid (e.g. 10-100 ug/ml), hydrocortisone (e.g. 50-500 ng/ml), EGF (e.g. 1-10 ng/ml), selenium (e.g. 1-10 ng/ml), ethanolamine (e.g. 1-10 ug/ml), insulin (e.g. 1-20 ug/ml), transferrin (e.g. 1-10 ug/ml) and aprotinin (e.g. 10-100 KIU/ml) (see, for example, Media-GE2). The media may further comprise VEGF (e.g. 5-50 ng/ml) (see, for example, Media-VGE2).

In one embodiment, the second layer comprises at least 1, 2, 3, 4, 5, 6, 7, 8, 9, 10 or more layers of oral keratinocytes.

In one embodiment, the method further comprises culturing the three-dimensional cell composition at air-liquid interface. This may be performed in deep-well plates for between 1-35 days (e.g. 3-8 days or 10-21 days) for keratinocyte stratification, differentiation and maturation. The media may comprise a 1:1 mix of ESFM and KSFM supplemented with 0.5% fetal bovine serum (FBS), L-ascorbic acid (e.g. 10-100 ug/ml), hydrocortisone (e.g. 50-500 ng/ml), selenium (e.g. 1-10 ng/ml), ethanolamine (e.g. 1-10 ug/ml), insulin (e.g. 1-20 ug/ml), transferrin (e.g. 1-10 ug/ml) and aprotinin (e.g. 10-100 KIU/ml). The media may further comprise VEGF (e.g. 5-50 ng/ml).

In one embodiment, the method further comprises culturing the three-dimensional cell composition at air-liquid interface. This may be performed in deep-well plates for between 1-35 days (e.g. 3-8 days or 10-21 days) for keratinocyte stratification, differentiation and maturation. The media may comprise a 1:1 mix of ESFM and KSFM supplemented with human serum (0.5-5%), human plasma lysate (0.5-5%), human serum albumin (0.5-5%), L-ascorbic acid (e.g. 10-100 ug/ml), hydrocortisone (e.g. 50-500 ng/ml), selenium (e.g. 1-10 ng/ml), ethanolamine (e.g. 1-10 ug/ml), insulin (e.g. 1-20 ug/ml), transferrin (e.g. 1-10 ug/ml) and aprotinin (e.g. 10-100 KIU/ml) (see, for example, Media-GE3). The media may further comprise VEGF (e.g. 5-50 ng/ml) (see, for example, Media VGE3).

The term Air-Liquid Interface (ALI) refers to the culture of cells such that their basal membrane is in contact with, or submerged in, liquid and their apical membrane is in contact with air. Advantageously, the oral keratinocytes consequently demonstrate apical-basal polarity in their differentiation resulting in the development of functional keratinised surfaces as seen in vivo. The period of ALI culture may range from 1 day to 35 days. The period of ALI culture may range from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 days. In one embodiment, the period of ALI culture is about 10-21 days. In another embodiment, the period of ALI culture is about 3-8 days. Intriguingly, the period of ALI culture may affect the type of tissue that is generated. For example, an ALI culture period of 10-21 days was found to lead to the formation of a gingival epithelial equivalent layer while an ALI culture period of 3-8 days was found to lead to the formation of an oral mucosa epithelial equivalent layer.

In one embodiment, the second layer of the three-dimensional cell composition (i.e. the gingival epithelial equivalent layer) comprises at least three layers of oral keratinocytes covered by a superficial corneal layer.

In one embodiment, the second layer of the three-dimensional cell composition (i.e. the oral mucosa epithelial equivalent layer) comprises at least three layers of oral keratinocytes without a superficial corneal layer.

Disclosed herein is a three-dimensional cell composition obtained according to a method as defined herein.

Disclosed herein is a three-dimensional cell composition comprising a) a first layer comprising a PEG-fibrin support matrix containing oral fibroblasts suspended within the support matrix, wherein the support matrix comprises fibrin, a modifier and oral fibroblasts and b) a second layer comprising oral keratinocytes. The second layer may be disposed on a surface of the first layer.

The three-dimensional cell composition as defined herein may be used in tissue engineering or tissue regenerative applications including but not limited to gingival, periodontal, oral mucosa, skin, esophagus, vagina and urethra regenerative applications. The cell composition can be implanted, grafted, or injected into animals or humans for tissue regeneration or replacement of defective tissue.

Provided herein is a tissue graft comprising a three-dimensional cell composition as defined herein.

Disclosed herein is a three-dimensional cell composition as defined herein for use as a medicament.

Disclosed herein is a method of treating a gum disease or condition, the method comprising administering a three-dimensional cell composition as defined herein to a subject.

Disclosed herein is a three-dimensional cell composition as defined herein for use in treating a gum disease or condition.

Disclosed herein is the use of a three-dimensional cell composition as defined herein in the manufacture of a medicament for treating a gum disease or condition.

The terms “treating”, “treatment” and the like, are used interchangeably herein to mean relieving, reducing, alleviating, ameliorating or otherwise inhibiting the condition, including one or more symptoms of the condition.

The three-dimensional cell composition may be used for treating a subject. The terms “patient”, “subject”, “host” or “individual”, used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the phylum Chordata including primates (e.g., humans, monkeys and apes, and includes species of monkeys such from the genus Macaca (e.g., cynomolgous monkeys such as Macaca fascicularis, and/or rhesus monkeys (Macaca mulatta) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), marine mammals (e.g., dolphins, whales), reptiles (snakes, frogs, lizards etc.), and fish. In one embodiment, the subject is human.

Disclosed herein is the use of a three-dimensional cell composition as defined herein for in vitro testing. The three-dimensional cell composition may be used for in vitro testing of a compound. The three-dimensional cell composition may also, for example, be used for in vitro drug testing, consumer-care product testing, studies on transmucosal permeation, drug delivery, ageing, host-microbiomeinteraction, host-biomaterial interaction, host-implant interaction or drug efficacy, and assays on safety, toxicity or biocompatibility.

The three-dimensional cell composition may, for example, be used for testing the mucosal irritation, mucosal corrosion and barrier disruption potential of actives and formulations of consumer-care products including but not limited to mouthwash, toothpastes, bleaching agents, mouth fresheners, oral irrigators, disclosing tablets or solutions, oral gels, oral sprays, dental cements, dental adhesives, etching agents and denture fixatives. The three-dimensional cell composition may be used for investigating the biocompatibility (acute toxicity) of dental therapeutics and biomaterials including but not limited to dental cements, dental composite, dental adhesive, denture adhesive, etching agents, bleaching agents.

In one embodiment, the three-dimensional cell composition is used for transmucosal delivery of dental anaesthetic (such as lidocaine hydrochloride or articaine hydrochloride).

In one embodiment, the three-dimensional cell composition is used for aging studies.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

### Example 1

Methodology

1. Fabrication of Mucosal Matrix (Lamina Propria Equivalent):

To fabricate the lamina propria equivalent, gingival or oral fibroblasts are embedded within a fibrin-based matrix. The fibrin-based matrix consists of three components:
Solution-A: (5 parts)
   Human plasma fibrinogen (10-80 mg/ml): 4 parts
   Poly(ethylene oxide), 4-arm, succinimidyl glutarate terminated (10 mg/ml): 1 part
Solution-B: (16 parts)
   Human Thrombin (100-400 UN/ml): 1 part
   Calcium chloride (40 mM): 8 parts
   Distilled water: 7 parts
Solution-C: (11 parts)
   Fibroblast suspension (1-20×10⁶ cells/ml): 1 part
   Opti-MEM: 10 parts

The total volumes of each component is dependent on the number of tissue equivalents and the total volume of fibrin-based matrix to be prepared.

Firstly, the components of the solution-A are mixed together and incubated at 37° C. for 30 mins. Second, the components of the solution-B are mixed together and placed on ice. Thirdly, the gingival or oral fibroblasts are dissociated from the culture flasks and resuspended to the desired final concentration. Using the fibroblast-cell suspension, the solution-C is prepared. After the 30 min incubation time, solution-A and solution-C are mixed together. Then solution-B is added to the solution-A+C mix and immediately pipetted into a cell culture insert. After 15-30 minutes of gelation period, culture media (Media-GE1) is added to each insert and the plates placed in the incubator.

Media-GE1 is composed of endothelial serum-free media (ESFM, GIBCO) supplemented with human serum (0.5-5%), human plasma lysate (0.5-5%), human serum albumin (0.5-5%), L-ascorbic acid (10-100 ug/ml), hydrocortisone (50-500 ng/ml), basic fibroblast growth factor (bFGF, 1-20 ng/ml), selenium (1-10 ng/ml), ethanolamine (1-10 ug/ml), insulin (1-20 ug/ml), transferrin (1-10 ug/ml) and aprotinin (10-100 KIU/ml).

2. Fabrication of Full-Thickness Gingival Equivalents (Gingiva-FT):

After 2-6 days of culture of lamina propria equivalents, gingival or oral keratinocytes are seeded on top of the matrix and cultured using Media-GE2 for 1-3 days. Then, the 3D cultures are transferred to deep-well plates and cultured at air-liquid interface using Media-GE3 for 10-21 days for keratinocyte stratification, differentiation and maturation.

Media-GE2 is composed of 1:1 mix of ESFM and keratinocyte serum-free media (KSFM, GIBCO) supplemented with human serum (0.5-5%), human plasma lysate (0.5-5%), human serum albumin (0.5-5%), L-ascorbic acid (10-100 ug/ml), hydrocortisone (50-500 ng/ml), EGF (1-10 ng/ml), selenium (1-10 ng/ml), ethanolamine (1-10 ug/ml), insulin (1-20 ug/ml), transferrin (1-10 ug/ml) and aprotinin (10-100 KIU/ml).

Media-GE3 is composed of 1:1 mix of ESFM and KSFM supplemented with human serum (0.5-5%), human plasma lysate (0.5-5%), human serum albumin (0.5-5%), L-ascorbic acid (10-100 ug/ml), hydrocortisone (50-500 ng/ml), selenium (1-10 ng/ml), ethanolamine (1-10 ug/ml), insulin (1-20 ug/ml), transferrin (1-10 ug/ml) and aprotinin (10-100 KIU/ml).

3. Fabrication of Vascularized Mucosal Matrix (Vascularized Lamina Propria Equivalent):

To fabricate the vascularized lamina propria equivalent, gingival or oral fibroblasts and endothelial cells are embedded within a fibrin-based matrix. The fibrin-based matrix consists of three components:
Solution-A: (5 parts)
   Human plasma fibrinogen (10-80 mg/ml): 4 parts
   Poly(ethylene oxide), 4-arm, succinimidyl glutarate terminated (10 mg/ml): 1 part
Solution-B: (16 parts)
   Human Thrombin (100-400 UN/ml): 1 part
   Calcium chloride (40 mM): 8 parts
   Distilled water: 7 parts
Solution-C: (11 parts)
   Fibroblast cell suspension (1-20×10⁶ cells/ml): 1 part
   Endothelial cell suspension (1-20×10⁶ cells/ml): 6 parts
   ESFM: 5 parts

The total volumes of each component is dependent on the number of tissue equivalents and the total volume of fibrin-based matrix to be prepared.

Firstly, the components of the solution-A are mixed together and incubated at 37° C. for 30 mins·Second, the components of the solution-B are mixed together and placed on ice. Thirdly, the gingival or oral fibroblasts and endothelial cells are dissociated from the culture flasks and resuspended to the desired final concentration. Using the cell suspension obtained, the solution-C is prepared. After the 30 min incubation time, solution-A and solution-C are mixed together. Then solution-B is added to the solution-A+C mix and immediately pipetted into a cell culture insert. After 15-30 minutes of gelation period, culture media (Media-VGE1) is added to each insert and the plates placed in the incubator.

Media-VGE1 is composed of ESFM supplemented with human serum (0.5-5%), human plasma lysate (0.5-5%), human serum albumin (0.5-5%), L-ascorbic acid (10-100 ug/ml), hydrocortisone (50-500 ng/ml), vascular endothelial growth factor (VEGF, 5-50 ng/ml), epidermal growth factor (EGF, 1-10 ng/ml), basic fibroblast growth factor (bFGF, 1-20 ng/ml), selenium (1-10 ng/ml), ethanolamine (1-10 ug/ml), insulin (1-20 ug/ml), transferrin (1-10 ug/ml) and aprotinin (10-100 KIU/ml).

4. Fabrication of Full-Thickness, Vascularized Gingival Equivalents (Gingiva-FT-V):

After 2-8 days of culture of vascularized lamina propria equivalents, gingival or oral keratinocytes are seeded on top of the matrix and cultured using Media-VGE2 for 1-3 days. Then, the 3D cultures are transferred to deep-well plates and cultured at air-liquid interface using Media-VGE3 for 10-21 days for keratinocyte stratification, differentiation and maturation.

Media-VGE2 is composed of 1:1 mix of ESFM and keratinocyte serum-free media (KSFM, GIBCO) supplemented with human serum (0.5-5%), human plasma lysate (0.5-5%), human serum albumin (0.5-5%), L-ascorbic acid (10-100 ug/ml), hydrocortisone (50-500 ng/ml), VEGF (5-50 ng/ml), EGF (1-10 ng/ml), selenium (1-10 ng/ml), ethanolamine (1-10 ug/ml), insulin (1-20 ug/ml), transferrin (1-10 ug/ml) and aprotinin (10-100 KIU/ml).

Media-VGE3 is composed of 1:1 mix of ESFM and KSFM supplemented with human serum (0.5-5%), human plasma lysate (0.5-5%), human serum albumin (0.5-5%), L-ascorbic acid (10-100 ug/ml), hydrocortisone (50-500 ng/ml), VEGF (5-50 ng/ml), selenium (1-10 ng/ml), ethanolamine (1-10 ug/ml), insulin (1-20 ug/ml), transferrin (1-10 ug/ml) and aprotinin (10-100 KIU/ml).

5. Fabrication of Full-Thickness Oral Mucosa Equivalents (Oral Mucosa-FT):

After 2-6 days of culture of lamina propria equivalents, oral keratinocytes are seeded on top of the matrix and cultured using Media-OME2 for 1-3 days. Then, the 3D cultures are transferred to deep-well plates and cultured at air-liquid interface using Media-OME3 for 3-8 days for keratinocyte stratification, differentiation and maturation.

Media-OME2 is composed of 1:1 mix of ESFM and keratinocyte serum-free media (KSFM, GIBCO) supplemented with human serum (0.5-5%), human plasma lysate (0.5-5%), human serum albumin (0.5-5%), L-ascorbic acid (10-100 ug/ml), hydrocortisone (50-500 ng/ml), EGF (1-10 ng/ml), selenium (1-10 ng/ml), ethanolamine (1-10 ug/ml), insulin (1-20 ug/ml), transferrin (1-10 ug/ml) and aprotinin (10-100 KIU/ml).

Media-OME3 is composed of 1:1 mix of ESFM and KSFM supplemented with human serum (0.5-5%), human plasma lysate (0.5-5%), human serum albumin (0.5-5%), L-ascorbic acid (10-100 ug/ml), hydrocortisone (50-500 ng/ml), selenium (1-10 ng/ml), ethanolamine (1-10 ug/ml), insulin (1-20 ug/ml), transferrin (1-10 ug/ml) and aprotinin (10-100 KIU/ml).

6. Fabrication of Full-Thickness Vascularized Oral Mucosa Equivalents (Oral Mucosa-FT-V):

After 2-8 days of culture of vascularized lamina propria equivalents, oral keratinocytes are seeded on top of the matrix and cultured using Media-VOME2 for 1-3 days. Then, the 3D cultures are transferred to deep-well plates and cultured at air-liquid interface using Media-VOME3 for 3-8 days for keratinocyte stratification, differentiation and maturation.

Media-VOME2 is composed of 1:1 mix of ESFM and keratinocyte serum-free media (KSFM, GIBCO) supplemented with human serum (0.5-5%), human plasma lysate (0.5-5%), human serum albumin (0.5-5%), L-ascorbic acid (10-100 ug/ml), hydrocortisone (50-500 ng/ml), VEGF (5-50 ng/ml), EGF (1-10 ng/ml), selenium (1-10 ng/ml), ethanolamine (1-10 ug/ml), insulin (1-20 ug/ml), transferrin (1-10 ug/ml) and aprotinin (10-100 KIU/ml).

Media-VOME3 is composed of 1:1 mix of ESFM and KSFM supplemented with human serum (0.5-5%), human plasma lysate (0.5-5%), human serum albumin (0.5-5%), L-ascorbic acid (10-100 ug/ml), hydrocortisone (50-500 ng/ml), VEGF (5-50 ng/ml), selenium (1-10 ng/ml), ethanolamine (1-10 ug/ml), insulin (1-20 ug/ml), transferrin (1-10 ug/ml) and aprotinin (10-100 KIU/ml).

Results

1. Gross Appearance

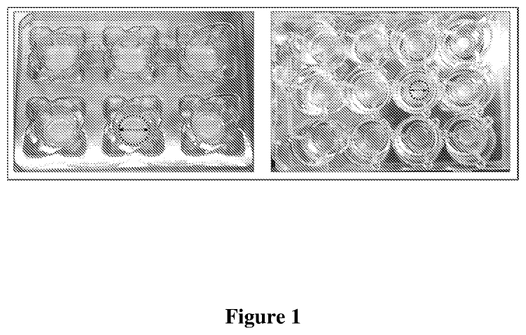

Fibrin-based matrix enabled the fabrication of contraction-free gingival tissue equivalents. FIG. 1 shows the gingival tissue equivalents fabricated within 6-well and 12-well insert formats. The 6-well insert format provides gingival tissue equivalents of 22 mm in diameter and 3.8 cm² in area. Similarly, the 12-well insert format provides gingival tissue equivalents of 10 mm in diameter and 0.78 cm² in area.

2. Characterization of Gingiva-FT:

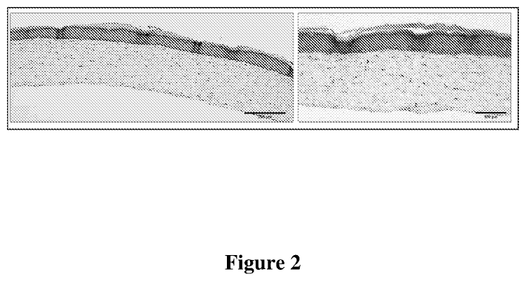

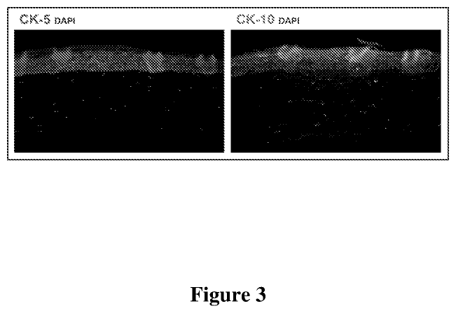

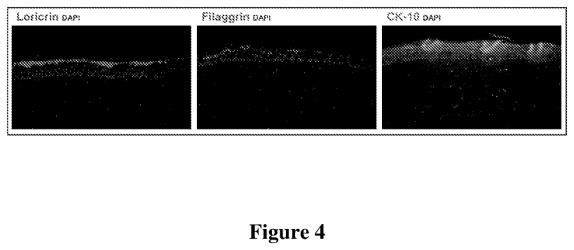

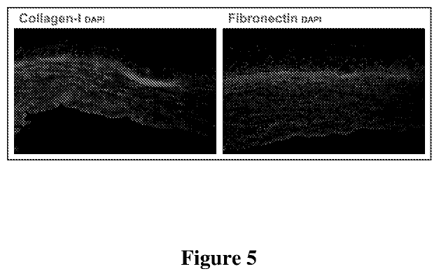

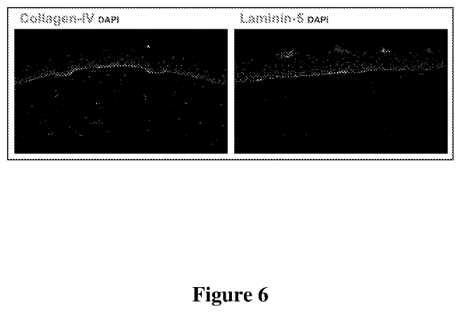

The full-thickness gingival tissue equivalents (Gingiva-FT) exhibit resemblance to native gingiva in terms of morphological and functional characteristics. Haematoxylin-eosin stained images demonstrate the presence of keratinized stratified squamous epithelium consisting of the basal, spinous, granular and cornified layers on a gingival fibroblast-populated lamina propria matrix, similar to the native human gingival tissue (FIG. 2). Additionally, the stratification of the Gingiva-FT tissues demonstrates the expression of cytokeratin-5 (CK-5) across the basal and suprabasal layers, and CK-10 expression in the suprabasal layers (spinous, granular and cornified layers) (FIG. 3). The maturity of the Gingiva-FT are demonstrated by the expression of epithelial differentiation markers: CK-10 expression in the suprabasal layers, while loricrin and filaggrin are expressed strongly in the granular-cornified layer (FIG. 4). Further, extracellular matrix proteins like collagen-1 and fibronectin are strongly expressed in the lamina propria matrix (FIG. 5). The junction between the gingival epithelium and the underlying lamina propria is demonstrated by the expression of basement membrane proteins (collagen type IV and laminin-5) (FIG. 6). Overall, these histological features confirm the formation of full-thickness gingival tissues in vitro similar to native human gingival tissues.

3. Characterization of Gingiva-FT-V:

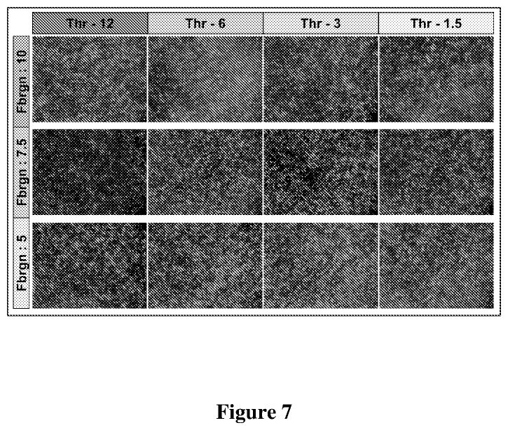

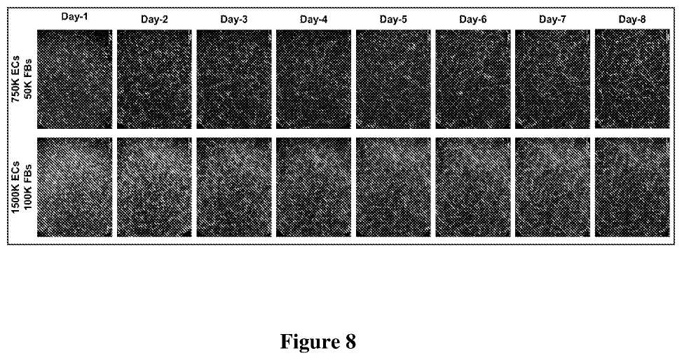

For the fabrication of full-thickness vascularized gingival tissue equivalents (Gingiva-FT-V), the conditions for the assembly of microvascular networks were first optimized for the construction of vascularized lamina propria equivalents. FIG. 7, 8 demonstrates the formation of microvascular networks in the vascularized lamina propria equivalents fabricated using varying concentrations of fibrinogen, thrombin and cellular parameters (number of endothelial cells per unit volume of the matrix and the ratio of endothelial cells to fibroblasts). Further, FIG. 8 demonstrates the kinetics of microvascular network formation over an 8-day culture period. The microvascular networks form as early as day-3 and are stable over long-term culture.

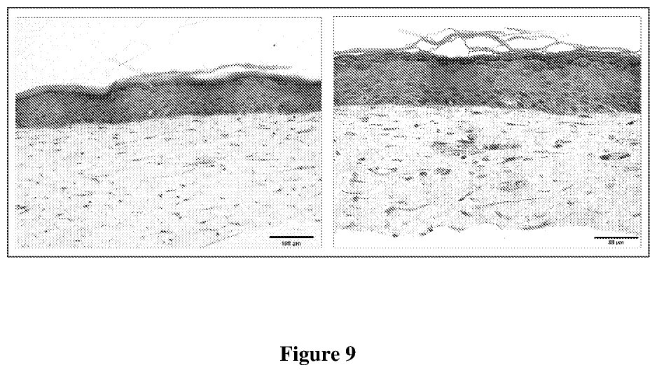

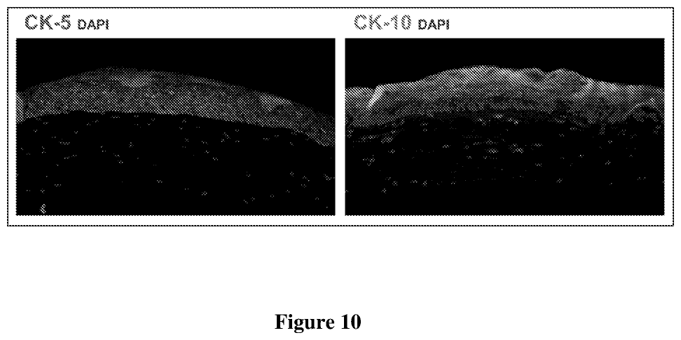

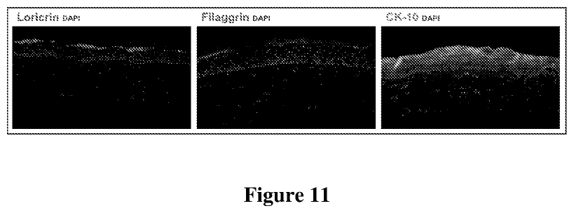

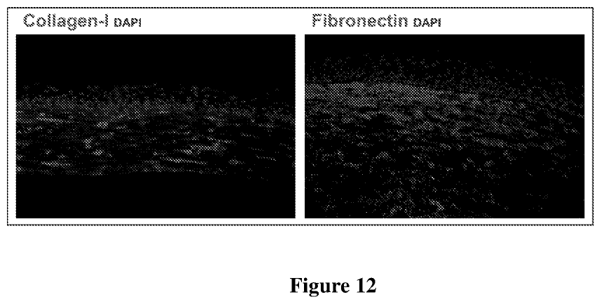

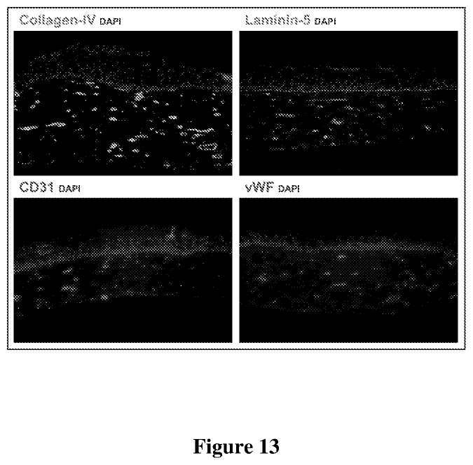

The full-thickness vascularized gingival tissue equivalents (Gingiva-FT-V) exhibit resemblance to native gingiva in terms of the presence of vasculature and the morphological features. Haematoxylin-eosin stained images demonstrate the presence of keratinized stratified squamous epithelium consisting of the basal, spinous, granular and cornified layers on a vascularized lamina propria matrix, similar to the native human gingival tissue (FIG. 9). The stratification of the Gingiva-FT-V tissues demonstrate the expression of cytokeratin-5 (CK-5) across the basal and suprabasal layers, and CK-10 expression in the suprabasal layers (spinous, granular and cornified layers) (FIG. 10). The maturity of the Gingiva-FT-V are demonstrated by the expression of epithelial differentiation markers: CK-10 expression in the suprabasal layers, while loricrin and filaggrin are expressed strongly in the granular-cornified layer (FIG. 11). Further, extracellular matrix proteins like collagen-1 and fibronectin are expressed in the lamina propria matrix (FIG. 12). The junction between the gingival epithelium and the underlying lamina propria is demonstrated by the expression of basement membrane proteins (collagen type IV and laminin-5) (FIG. 13). Importantly, the presence of lumenized blood vessels within the lamina propria is demonstrated by the expression of CD31, vWF, collagen-IV and laminin-5 (FIG. 13). These markers label the blood vessels, which can be seen as circular to elongated structures with a lumen. Overall, these histological features confirm the formation of full-thickness vascularized gingival tissues in vitro similar to native human gingival tissues.

### Example 2

Validation of Gingival Tissue Constructs for Downstream Applications

Oral-care and dental-care products are commonly used daily for personal care and therapeutic reasons. Before these products can reach the market, they are tested for their safety, toxicity and biocompatibility such as mucosal irritation and corrosion studies.

Similarly, during the product development phase, it is essential to identify the mucosal toxicity, biocompatibility and efficacy of novel actives, excipients and drug/product formulations. The 3D cultured gingival tissue equivalents of the present invention may be used widely as an alternative to animal experiments in toxicity and efficacy studies of oral and dental-care products, in drug permeation studies, in host-microbiome studies to study interaction between gingival tissues and oral bacteria, and as a tissue engineered grafts for periodontal or oral mucosal regeneration related applications. Some of the validation studies for various downstream applications are provided below as case study examples. These full-thickness gingival tissue equivalents have been used for applications related to biocompatibility testing, drug permeation studies, toxicity testing, host-microbiome studies and periodontal regeneration.

A. Mucosal Irritation, Corrosion and Barrier Integrity Studies

Mucosal irritation refers to the reversible damage to the mucosal tissues caused following the application of the test substance. On the other hand, mucosal corrosion refers to irreversible tissue damage upon application of the test substance. The potential of chemical-induced irritation and corrosion are important considerations in safety evaluation of oral-care, dental-care and pharmaceutical products intended for human use. Hence, understanding the potential of oral and dental-care products, actives and excipients for mucosal irritation and corrosion is important for hazard identification and reduce potential risks.

1) Use of Gingiva-FT for Investigating the Potential of Mouthwashes for Mucosal Irritation on Intact Mucosal Tissues

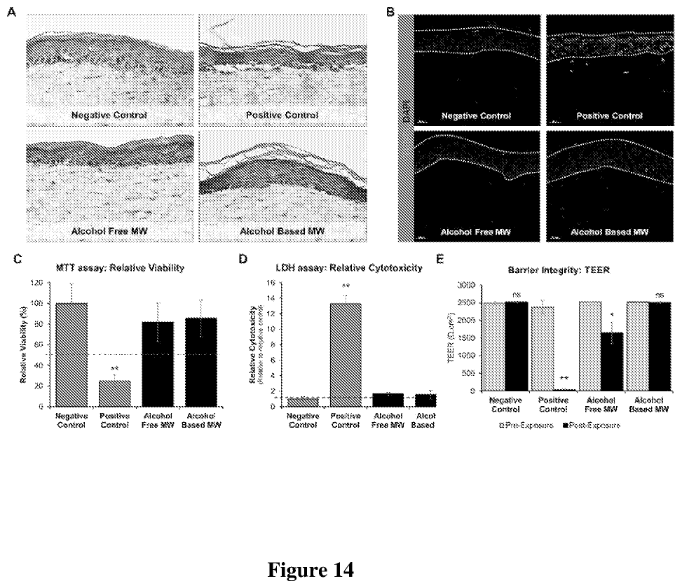

To evaluate the use of 3D cultured gingival tissue equivalents of the present invention for mucosal irritation, we investigated the impact of 2 commercially available mouthwashes. To mimic the actual use case scenario, gingival tissue equivalents (surface area 0.78 cm²) were exposed to 130 μl alcohol-free (Listerine® Gum care Zero) or alcohol-based (Listerine® Cool Mint) mouthwashes for 30 secs, washed, and cultured. After 10 hours, the tissues were re-exposed to the respective mouthwash, washed and cultured for 24 hours. Phosphate buffered saline (PBS) and 1% sodium lauryl sulphate was used as negative and positive controls respectively. Based on the OECD TG439 guidelines, 50% cellular viability was set as threshold for classification of mucosal irritants. Compared to the negative controls, the gingival equivalents exposed to positive control exhibited significant tissue disruption, TUNEL positivity (apoptosis/cell death), reduced cellular viability (<50%) and markedly increased cytotoxicity (FIG. 14). On the other hand, both the mouthwash types showed minimal evidence of epithelial disruption and barrier integrity (FIG. 14A). TUNEL staining demonstrated the absence of TUNEL-positive cells in the samples treated with mouthwashes similar to negative control (FIG. 14B). Based on MTT and LDH assays both mouthwashes demonstrated minimal impact on cellular viability and cytotoxicity (FIG. 14C,D). These results indicate that both alcohol-free and alcohol-based mouthwash used in this case study had minimal mucosal irritation potential compared to positive control. However, assessment of barrier integrity using trans epidermal electrical resistance (TEER) showed a significant reduction in TEER in samples exposed to alcohol-free mouthwash (FIG. 14E). This could be due to the use of sodium lauryl sulphate in the alcohol-free mouthwash used in this study. Thus, Gingiva-FT tissue constructs provide opportunity to study mucosal irritation and barrier integrity of actives and excipients used in oral and dental-care products.

2) Use of Lamina Propria Equivalents for Investigating the Potential of Mouthwashes for Mucosal Irritation on Ulcerated Mucosal Tissues

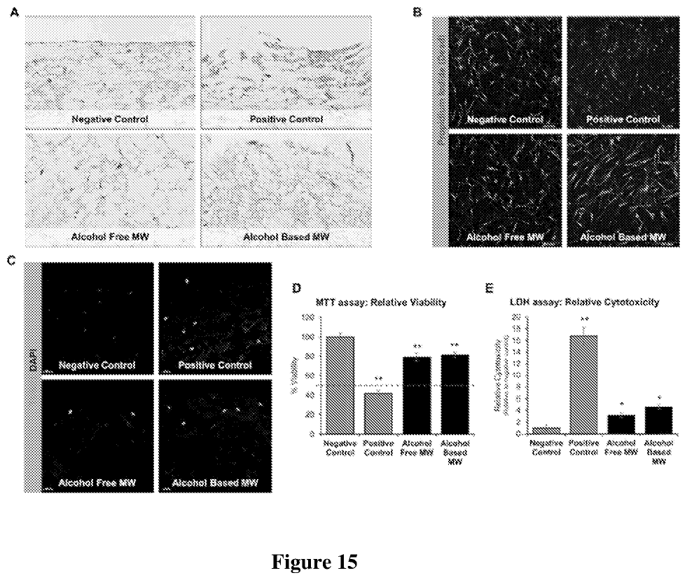

Oral ulcers are one of the most common disturbances in the oral cavity. Since, the oral ulcers is devoid of the overlying epithelium, the barrier function provided by the epithelium is lost. Hence, oral ulcers could be sensitive to external agents. To evaluate the use of 3D cultured lamina propria equivalents as a model of oral ulcers, we investigated the impact of 2 commercially available mouthwashes for mucosal irritation. To mimic the actual use case scenario, gingival tissue equivalents (surface area 0.78 cm²) were exposed to 130 μl alcohol-free (Listerine® Gum care Zero) or alcohol-based (Listerine® Cool Mint) mouthwashes for 30 secs, washed, and cultured. After 10 hours, the tissues were re-exposed to the respective mouthwash, washed and cultured for 24 hours. Phosphate buffered saline (PBS) and 1% sodium lauryl sulphate was used as negative and positive controls respectively. Based on the OECD TG439 guidelines, 50% cellular viability was set as threshold for classification of mucosal irritation. Compared to the negative controls, the gingival equivalents exposed to positive control exhibited significant tissue disruption, TUNEL positivity (apoptosis/cell death), reduced cellular viability (<50%) and markedly increased cytotoxicity (FIG. 15). On the other hand, both the mouthwash types showed some evidence of tissue disruption (FIG. 15). LIVE/DEAD staining of the mouthwash treated samples showed the presence of viable cells and a few dead cells. However, TUNEL staining showed the increased presence of TUNEL-positive cells in the mouthwash treated samples, suggesting early damage or induction of cell death (FIG. 15B,C). Based on MTT and LDH assays both mouthwashes demonstrated significant decrease in cellular viability and increased LDH cytotoxicity. However, these lowered viability levels were within acceptable limits (>50% based on OECD TG439 guidelines) suggesting the mouthwashes lack mucosal irritation potential upon short-term (30 s) exposure (FIG. 15D,E). Thus, the lamina propria equivalents that is used as a base matrix to fabricate Gingiva-FT and Gingiva-FT-V tissue constructs could be used as oral mucosal ulcer model. This provides opportunity to understand mucosal irritation potential of actives and excipients used in oral and dental-care products with respect to oral ulcers.

3) Use of Gingiva-FT for Investigating the Potential of Mouthwashes for Mucosal Corrosion

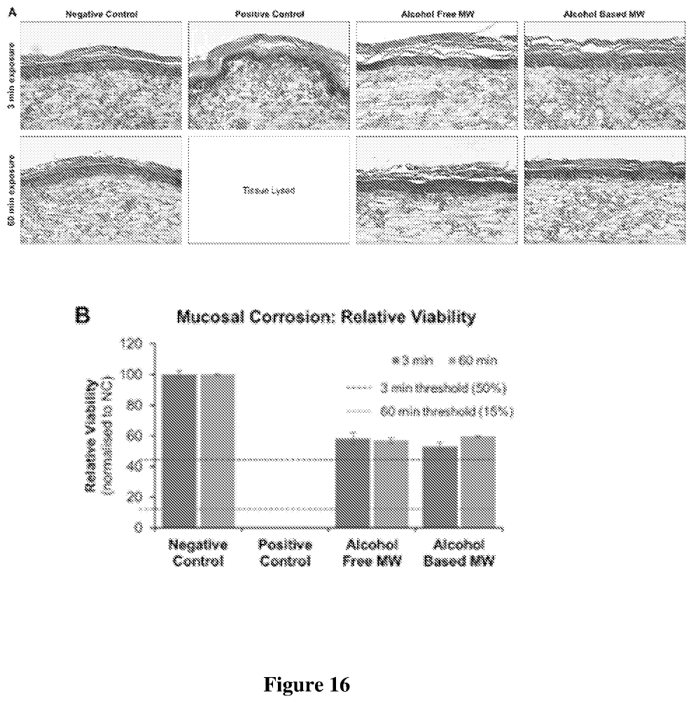

Some excipients used to incorporate the actives and formulate oral/dental care products and pharmaceuticals, have the potential to cause mucosal corrosion similar to caustic substances like acids and bases. To evaluate the use of 3D cultured gingival tissue equivalents of the present invention for mucosal corrosion, we adopted the OECD TG431 guidelines to investigate the mucosal corrosion potential of 2 commercially available mouthwashes. Based on the OECD TG431 guidelines, gingival tissue equivalents (surface area 0.78 cm²) were exposed to 65 μl alcohol-free (Listerine® Gum care Zero) or alcohol-based (Listerine® Cool Mint) mouthwashes for 3 and 60 mins, washed, and evaluated for cellular viability. Phosphate buffered saline (PBS) and 37% phosphoric acid was used as negative and positive controls respectively. Based on the OECD TG431 guidelines, 50% and 15% cellular viability was set as thresholds for 3 min and 60 min exposures respectively. Compared to the negative controls, the gingival equivalents exposed to 37% phosphoric acid exhibited tissue disruption after 3 min exposure and almost complete lysis of the tissues after 60 min exposure (FIG. 16A). The tissues showed less than 5% cellular viability at 3 min and 60 min exposure, suggesting a classification as corrosive (category 1A) (FIG. 16A,B). Both the alcohol-free and alcohol-based mouthwashes showed significant reduction in relative cellular viability after 3 min and 60 min exposure (FIG. 16A,B). However, the cellular viability was above the threshold levels, suggesting a classification of both mouthwash as non-corrosive. Thus, the Gingiva-FT tissue constructs provide opportunity to study mucosal corrosion potential of actives and excipients used in oral and dental-care products and pharmaceuticals.

B. Drug Permeation & Drug Delivery Applications

Drug delivery through oral tissues is increasingly investigated by researchers and pharmaceuticals as oral tissues offer various advantages compared to systemic delivery. For instance, the absorption of drugs is faster through oral tissues compared to gut or skin tissues; it is not affected by digestive enzymes in the gut; can reach the blood stream directly; and hence, require low effective dosages.

1) Use of Gingiva-FT for Transmucosal Drug Permeation/Delivery Applications

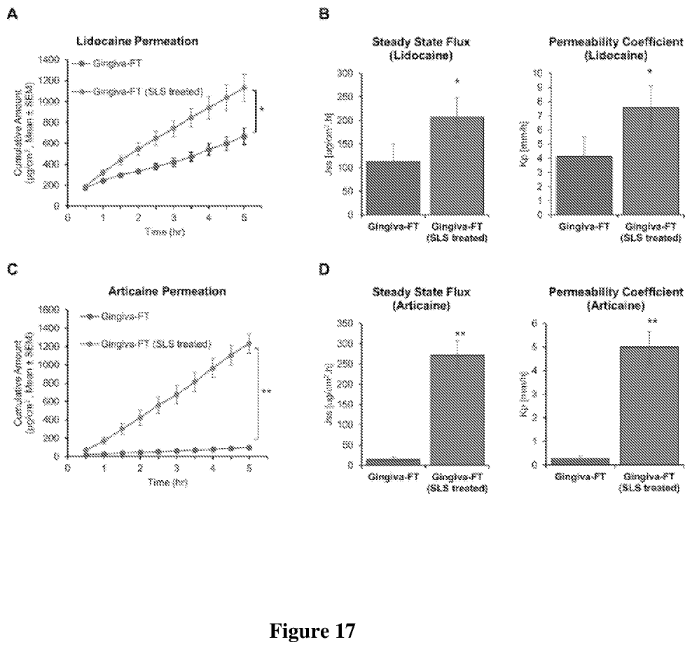

To validate the use of Gingiva-FT and Gingiva-FT-V tissues for transmucosal drug delivery applications, the permeation of model drugs such as dental anaesthetics were evaluated. Dental anaesthetics lidocaine hydrochloride and articaine hydrochloride were used as model drugs. A liquid suspension of 150 μl of Lidocaine hydrochloride (1.66 mg/ml) or articaine hydrochloride (3.32 mg/ml) representative of an infinite dose was loaded onto epithelial surface of the tissues, and the kinetics of permeation through the tissues over time were evaluated. To simulate diseased tissues with barrier disruption and/or formulations with permeation enhancers, some of the tissue constructs were treated with 1% sodium lauryl sulphate for 60 mins and gently washed prior to loading the dental anaesthetic. Analysis of the drug permeation over 5 hours showed the kinetics of permeation of lidocaine and articaine through intact and SLS-treated tissues (FIG. 17). Both lidocaine and articaine permeated through intact and SLS-treated tissues. However, the lidocaine and articaine permeation through SLS-treated tissues were significantly higher compared to intact tissues as demonstrated by permeation parameters like cumulative amount permeated, steady state flux and permeability coefficient (FIG. 17). These results demonstrate the potential to modulate the permeation kinetics of gingival tissue constructs and their use for drug permeation and drug delivery applications.

C. Applications Related to Biocompatibility Assessment of Dental Materials

Dental materials such as dental cements, dentures come in mid to long-term contact with the oral tissues. Hence, the ability of the dental material to function without causing adverse reaction to the oral tissues is important.

1) Biocompatibility (Acute Toxicity) of Dental Composites

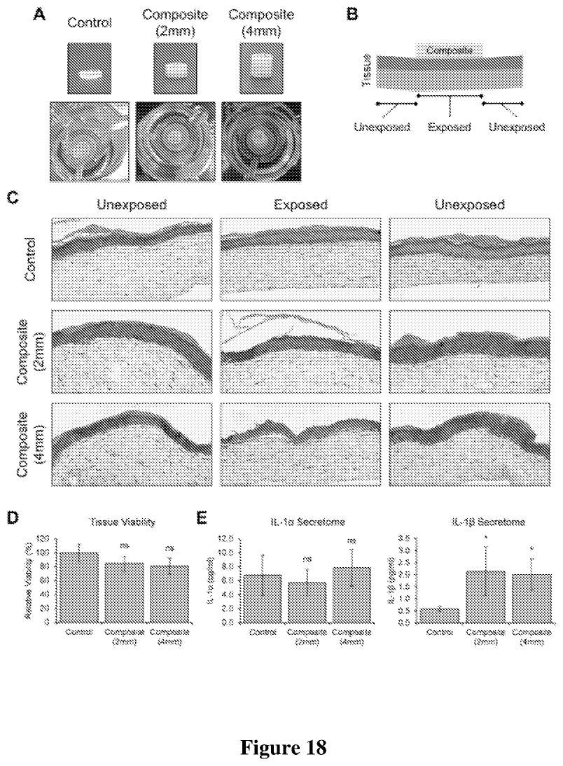

To validate the use of Gingiva-FT and Gingiva-FT-V tissues for dental material biocompatibility tests, discs of dental composite (Filtek Z350 XT, Cl body shade, 3M ESPE) were placed on top of the gingival constructs and cultured for 48 hours. A polyethylene plastic disc of 1 mm thickness was used as negative control. Based on manufacturer's recommendations, the ideal curing depth is 2 mm to avoid uncured resin at the deeper parts of the composite filling. Hence, composite discs of 2 mm and 4 mm thickness (that were cured under similar lighting conditions over 10 secs) were used to evaluate the acute toxicity potential of uncured resin (FIG. 18A). The tissue constructs used were 10 mm in diameter, while the composite discs were 5 mm in diameter. This uniquely allowed the ability to evaluate the impact of the composite resin on tissues in direct contact and on the surrounding unexposed tissues (FIG. 18B). Histological sections showed the disruption of the corneal layer in exposed regions of composite samples (FIG. 18C). Assessment of tissue viability using MTT assay showed a reduction in cellular viability, however the decrease was not statistically significant (FIG. 18D). Similarly, there was no difference in the IL-la, a cytokine release used as surrogate marker in mucosal irritation studies (FIG. 18E). However, another surrogate marker IL-1β representative of acute toxicity was significantly increased in both the composite-exposed tissues compared to the negative control (FIG. 18F). These results demonstrate the potential use of gingival and oral mucosal tissue constructs for biocompatibility assessment of dental materials and other biomaterials.

D. Aging Studies

Oral mucosal ageing has been associated with atrophic epithelium and impaired wound healing in gingival and periodontal tissues. Gingival fibroblasts play a central role in epithelial morphogenesis, scarless healing and healing following periodontal surgeries. However, the role of cellular aging of oral fibroblasts on gingival epithelial morphogenesis is poorly understood. Most studies rely on the use of in vitro monolayer cultures and animal models that poorly represent the human physiology. The close to human resemblance of Gingiva-FT and Gingiva-FT-V tissue constructs provide the opportunity to study oral mucosal and gingival ageing.

1). Use of Tissue Constructs for Oral Mucosal Ageing Studies

To study the impact of cellular ageing on oral mucosa, the culture protocol for the biofabrication of full-thickness oral mucosa equivalents (OME) were adapted from the protocol used for Gingiva-FT fabrication. Full-thickness OME were fabricated as follows: Firstly, the lamina propria equivalents was fabricated using oral mucosal fibroblasts embedded within the fibrin matrix (described earlier) in Media-GE1. After 2-6 days of culture of lamina propria equivalents, oral keratinocytes were seeded on top of the matrix and cultured using Media-OME2 for 1-3 days. Then, the 3D cultures are transferred to deep-well plates and cultured at air-liquid interface using Media-OME3 for 3-8 days for keratinocyte stratification, differentiation and maturation that would resemble the non-keratinised stratified squamous epithelium of lining oral mucosal tissues like buccal mucosa. To fabricate OME tissue constructs of young and aged OMEs, oral fibroblasts of early (passage 3-8) and late (passage 15-25) passages were used respectively.

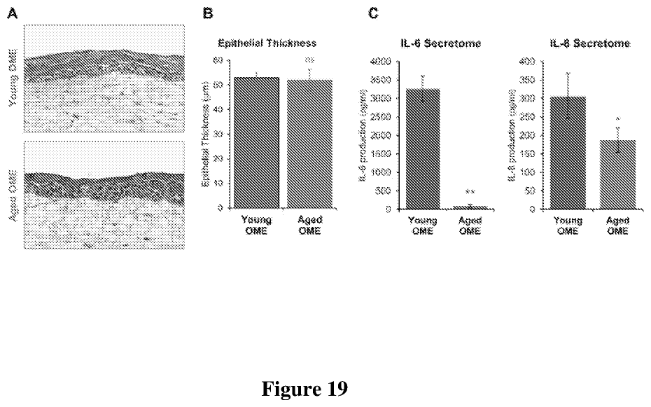

Histological evaluation and analysis of epithelial thickness showed no marked difference in the oral mucosal epithelial morphology and thickness between the young and aged oral mucosal phenotypes (FIG. 19A,B). This is similar to many clinical studies that demonstrate the absence of any observable difference in buccal mucosa tissues in the elderly. However, assessment of cytokine release profile showed significantly lower production of IL-6 and IL-8, key cytokines involved in innate immune response and wound healing (FIG. 19C). These results suggest a potentially impaired immune response and wound healing in the aged oral mucosal tissues. These studies show the potential to modulate the biofabrication methodologies to generate tissues representative of different phenotypes including young and aged oral mucosa.

2) Use of Gingiva-FT Tissue Constructs for Gingival Ageing Studies

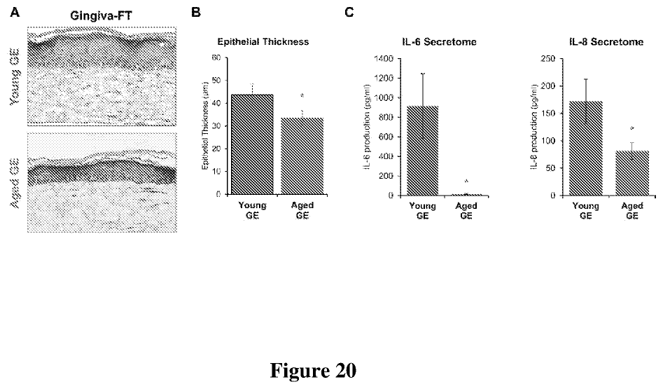

To study the impact of cellular ageing on gingiva, young and aged gingival constructs were biofabricated using the culture protocol for the biofabrication of Gingiva-FT with gingival fibroblasts of early (passage 3-8) and late (passage 15-25) passages respectively. Histological evaluation and analysis of epithelial thickness showed epithelial atrophy in the aged gingival tissues. This was further supported by the significantly reduced epithelial thickness measurements in the aged gingival tissues (FIG. 20A,B). Further, assessment of cytokine release profiles showed significantly lower production of IL-6 and IL-8, key cytokines involved in innate immune response and wound healing (FIG. 20C). These results suggest a potentially impaired barrier function, immune response and wound healing in the aged gingival tissues. These studies show the potential to modulate the biofabrication methodologies to generate tissues representative of different phenotypes including young and aged gingiva and their use in understanding and intervention of gingival ageing.

1. A method of preparing a three-dimensional cell composition, the method comprising the steps of:
a) forming a support matrix containing oral fibroblasts suspended within the support matrix by mixing fibrinogen, a modifier and oral fibroblast with thrombin;
b) incubating the support matrix in a cell culture media for a sufficient time to allow development of a first layer of the three-dimensional cell composition; and
c) seeding oral keratinocytes on a surface of the first layer and culturing the oral keratinocytes to form a second layer of the three-dimensional cell composition.

2. The method of claim 1, wherein the three-dimensional cell composition is an artificial gingival tissue.

3. The method of claim 1 or 2, wherein the first layer of the three-dimensional cell composition is a lamina propria equivalent layer.

4. The method of claim 1, wherein the three dimensional cell composition is an artificial oral mucosal tissue.

5. The method of any one of claims 1 to 4, wherein the oral fibroblasts are from gingival, periodontal ligament, buccal mucosa, palatal mucosa, labial mucosa, lingual mucosa or other oral mucosal surfaces.

6. The method of any one of claims 1 to 5, wherein the fibrinogen and modifier is cross-linked in the presence of thrombin.

7. The method of any one of claims 1 to 6, wherein the concentration of oral fibroblasts is from 1×10⁴ to 1×10⁶ cells/ml.

8. The method of any one of claims 1 to 7, wherein the support matrix further comprises endothelial cells.

9. The method of any one of claims 1 to 8, wherein the concentration of endothelial cells is from 1×10⁵ to 4×10⁶ cells/ml.

10. The method of any one of claims 1 to 9, wherein the fibrinogen is human fibrinogen.

11. The method of any one of claims 1 to 10, wherein the concentration of fibrinogen is 1.25 to 20 mg/ml.

12. The method of any one of claims 1 to 11, wherein the concentration of the modifier is 0.3 to 2.5 mg/ml.

13. The method of any one of claims 1 to 12, wherein the weight ratio of fibrinogen to modifier is about 4:1.

14. The method of any one of claims 1 to 13, wherein the modifier is a 2-arm, 4-arm or 8-arm PEG.

15. The method of claim 14, wherein the PEG is poly(ethylene oxide), 4-arm, succinimidyl glutarate terminated.

16. The method of any one of claims 1 to 15, wherein the thrombin is at a concentration of 3.125 IU/ml to 12.5 IU/ml.

17. The method of any one of claims 1 to 16, wherein the thrombin is human thrombin.

18. The method of any one of claims 1 to 17, wherein the step of forming the support matrix comprises forming the support matrix in a mold.

19. The method of any one of claims 1 to 18, wherein step b) comprises incubating the support matrix in a media supplemented with human serum (0.5-5%), human plasma lysate (0.5-5%), human serum albumin (0.5-5%), L-ascorbic acid (e.g. 10-100 ug/ml), hydrocortisone (e.g. 50-500 ng/ml), basic fibroblast growth factor (e.g. 1-20 ng/ml), selenium (e.g. 1-10 ng/ml), ethanolamine (e.g. 1-10 ug/ml), insulin (e.g. 1-20 ug/ml), transferrin (e.g. 1-10 ug/ml) and aprotinin (e.g. 10-100 KIU/ml).

20. The method of any one of claims 1 to 18, wherein step b) comprises incubating the support matrix in a media supplemented with human serum (0.5-5%), human plasma lysate (0.5-5%), human serum albumin (0.5-5%), L-ascorbic acid (e.g. 10-100 ug/ml), hydrocortisone (e.g. 50-500 ng/ml), VEGF (e.g. 5-50 ng/ml) and EGF (e.g. 1-10 ng/ml), basic fibroblast growth factor (e.g. 1-20 ng/ml), selenium (e.g. 1-10 ng/ml), ethanolamine (e.g. 1-10 ug/ml), insulin (e.g. 1-20 ug/ml), transferrin (e.g. 1-10 ug/ml) and aprotinin (e.g. 10-100 KIU/ml).

21. The method of claim 19 or 20, wherein the method comprises incubating the support matrix for 2 to 6 days to form the first layer of the three-dimensional cell composition.

22. The method of claim 1, wherein the second layer is a gingival epithelial equivalent layer.

23. The method of claim 1, wherein the second layer is a oral mucosal epithelial equivalent layer.

24. The method of claim 1, wherein the concentration of oral keratinocytes is from 1×10⁵ to 5×10⁵ cells/cm².

25. The method of claim 1, wherein the oral keratinocytes are from gingival, buccal mucosa, palatal mucosa, labial mucosa, lingual mucosa or other oral mucosal surfaces.

26. The method of any one of claims 1 to 25, wherein the method further comprises culturing the three-dimensional cell composition at air-liquid interface.

27. The method of claim 26, wherein the method comprises culturing the three-dimensional cell composition at air-liquid interface for 10-21 days or for 3-8 days.

28. A three-dimensional cell composition obtained according to a method of any one of claims 1 to 27.

29. A three-dimensional cell composition comprising a) a first layer comprising a PEG-fibrin support matrix containing oral fibroblasts suspended within the support matrix, wherein the support matrix comprises fibrin, a modifier and oral fibroblasts; and b) a second layer comprising oral keratinocytes.

30. A three-dimensional cell composition of claim 28 or 29 for use as a medicament.

31. Use of a three-dimensional cell composition of claim 28 or 29 in the manufacture of a medicament for treating a gum disease or condition.

32. Use of a three-dimensional cell composition of claim 28 or 29 in the manufacture of a medicament for regenerative therapy.

33. Use of a three-dimensional cell composition of claim 28 or 29 for in vitro testing.
